(19) United States
(12) Patent Application Publication
(10) Pub. No.: US 2008/0097599 A1
(43) Pub. Date: Apr. 24, 2008

(54) Posterior chamber Phakic Intraocular Lens

(76) Inventors: George W. Rozakis, North Olmsted, OH (US); Igor G. Valyunin, Hoboken, NJ (US); William Rozakis, Astoria, NY (US)

Correspondence Address: FULBRIGHT & JAWORSKI, LLP, 1301 MCKINNEY, SUITE 5100, HOUSTON, TX, 77010-3095, US

(21) Appl. No.: 11/857,476

(22) Filed: Sep. 19, 2007

Related U.S. Application Data
(60) Provisional application No. 60/852,944, filed on Oct. 19, 2006.

(51) Int. Cl. A61F 2/16

(52) U.S. Cl. 623/6.14

(57) ABSTRACT

The invention relates to a posterior chamber phakic intraocular lens with improved fluid flow between the anterior and posterior chambers of the eye. The fluid flow is accomplished by means of an opening situated in the center of the optical portion of the intraocular lens.

### CROSS-REFERENCE TO RELATED APPLICATIONS

This Application claims priority to provisional application 60/852,944 filed on Oct. 19, 2006.

### STATEMENT REGARDING FEDERALLY SPONSORED RESEARCH OR DEVELOPMENT

Not applicable.

### REFERENCE TO A COMPACT DISK APPENDIX

Not applicable.

### TECHNICAL FIELD

The present invention relates to posterior chamber, phakic intraocular lenses.

### BACKGROUND OF THE INVENTION

Most intraocular lenses implanted intermediate the iris and the crystalline lens are coupled with a surgical iridotomy. An iridotomy is an incision in the iris. The iridotomy has been used to ensure proper flow of aqueous with a phakic lens that covers the pupil. Aqueous flow occurs because the aqueous is produced by the ciliary body in the posterior chamber. The aqueous flows in front of the crystalline lens, through the pupil, and into the anterior chamber of the eye. The fluid then drains out of the eye through the trabecular meshwork at the base of the cornea. Although an iridotomy is a safe procedure, the procedure carries normal surgical risks and can increased the recovery time of a patient. An iridotomy may also change the fluid flow patterns within the eye.

U.S. Pat. No. 5,480,428 discloses a lens structure that eliminates the need for an iridotomy because an opening is defined by the optical body that protrudes through the iris at the pupil of the eye. The opening is described as allowing proper circulation of liquid within the eye. This lens structure includes a part-spherical optic that protrudes through the iris such that the engagement between the iris and optical maintains the position of the lens. The positioning elements of the lens rest directly against the crystalline lens in a manner that cause the iris to incline when the pupil contracts. The position of this lens within the eye is thus independent of the size of the hole defined by the optical portion and there is no reason to limit the size of the hole to inhibit fluid flow through the hole. A hole diameter of 0.4 mm to 0.5 mm is thus provided in the optical portion of the lens.

### BRIEF SUMMARY OF THE INVENTION

The invention provides a phakic lens structure having an opening disposed at the portion of the phakic lens to be positioned at the pupil of the eye. The opening is configured to allow a limited amount of fluid to flow directly through the lens. In combination with this opening, the optical body and positioning arms of the phakic lens are configured to allow the phakic lens to float off of the natural crystalline lens of the eye. The floating configuration promotes desirable fluid flow between the posterior surface of the phakic lens and the anterior surface of the crystalline lens. Flow is encouraged because the posterior surfaces of the positioning arms are not fixed against the anterior surface of the crystalline lens.

In one configuration of the invention, the size of the opening at the optical portion of the phakic lens is reduced to limit fluid flow through the phakic lens. The limited fluid flow is believed to promote a pocket of fluid intermediate the phakic lens and the crystalline lens which helps keep the phakic lens spaced from the crystalline lens. An opening having a maximum opening dimension (such as a diameter) of less than 0.4 mm is defined by a thin optical portion of the phakic lens. The combination of the small opening with the thin optic is believed to have a negligible impact on the user's vision and the power of the phakic lens.

Another configuration of the invention uses an elongated lens opening defined by the thinner portion of the optical portion of the lens body. The elongated opening may be used to influence fluid flow by encouraging fluid flow at the areas of the lens corresponding to the elongated direction of the opening.

Another configuration of the invention provides a non-circular opening defined the optical portion of the lens. The orientation of the non-circular opening is designed to indicate the position of the lens with respect to the eye. In one embodiment, an elongated opening is defined by the optical portion of the lens. The elongated opening is angled from bottom left to upper right when the lens is correctly positioned in the eye.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 depicts a schematic cross section of an eye with a phakic lens implanted in the posterior chamber in front of the crystalline lens without an iridotomy;

FIGS. 1A-C depict exemplary lens shapes in cross section using the hole configurations of the invention

FIGS. 2A-2F depict different cross sections for the lens walls that define the openings;

FIGS. 3 and 4 depict elongated openings that may be used with different types of phakic lenses;

FIG. 5 depicts a lens with an elongated opening that indicates the position of the lens within the eye.

Similar numbers refer to similar elements throughout the specification. The drawings are not to scale

### DETAILED DESCRIPTION OF THE INVENTION

An exemplary phakic lens 10 is depicted in the posterior chamber of an eye 12 having a cornea 14 and iris 16 and a crystalline lens 18. Lens 10 is implanted between the posterior surface of iris 16 and the anterior surface of crystalline lens 18 is a floating configuration that allows the lens to move within the posterior chamber of eye 12 without being wedged into the outer physical boundaries of the eye or wedged between the iris and crystalline lens.

Lens 10 is configured to direct a portion of the fluid produced by eye 12 to initially flow directly along the anterior surface of crystalline lens 18 as indicated by the reference line 20. This flow pattern is promoted by the use of an opening 26 in the central portion of lens 10. Fluid flow directly along the anterior surface of crystalline lens 18 is desirable because it mimics the flow the eye before a phakic lens is implanted. Such flow nourishes the eye and may be useful in preventing cataracts. Locating opening 26 in the central portion of lens 10 has been found to have little, if any, undesirable affect on the patient's vision.

An eye with an iridotomy allows fluid to bypass crystalline lens 18 by passing directly through the outer portion of iris 16 where the iridotomy holes are cut. When lens 10 is used without an iridotomy, fluid 20 helps to maintain lens 10 away from the anterior surface of crystalline lens 18. Fluid 20 continues flowing toward the central portion of lens 10 that defines the optical power of lens 10. Lens 10 defines a through opening 26 that allows the fluid 20 to pass from the posterior surface of lens 10 to the anterior surface 30. This fluid then passes through the pupil defined by the iris in the flow pattern as an eye without an implant.

In one configuration of the invention, opening 26 is sized to limit the fluid through the lens in order to use the fluid flow to position lens 10 within the posterior chamber of eye 12. Using a relatively small opening 26 appears to allow a small pocket of fluid to build between the anterior surface of crystalline lens 18 and posterior surface 28 of lens 10. Although lens 10 is still free to float within the chamber, this small pocket of fluid helps to create a lifting force that maintains spacing between lens 18 and lens 10. Fluid may still flow around the peripheral edge of lens 10 and through the pupil so that a flow path remains open.

An opening having a maximum (such as a diameter) dimension of less than 0.6 mm and preferably less than 0.4 mm may be used to create the desired pocket of fluid.

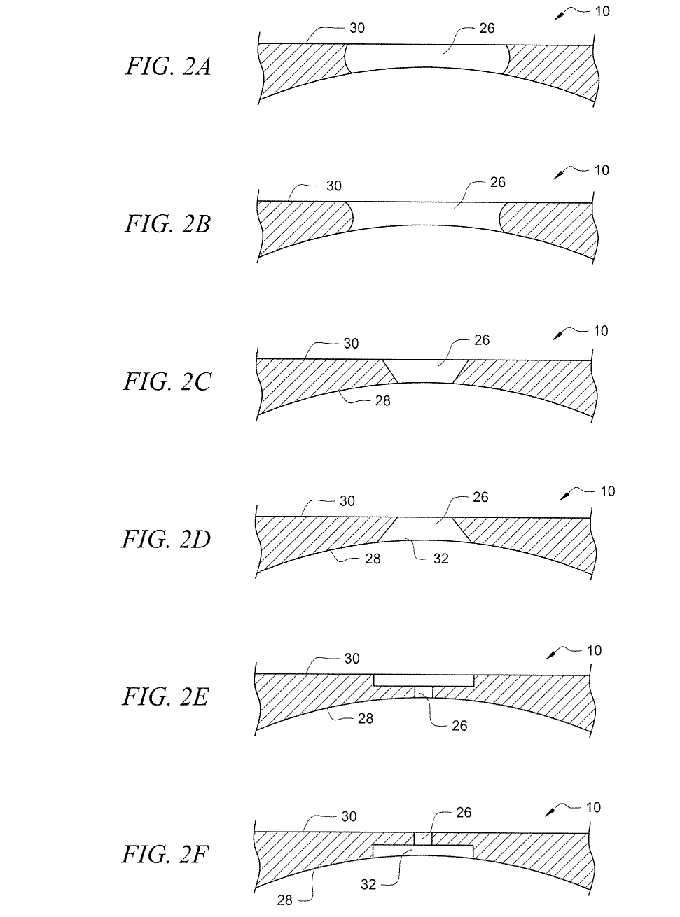

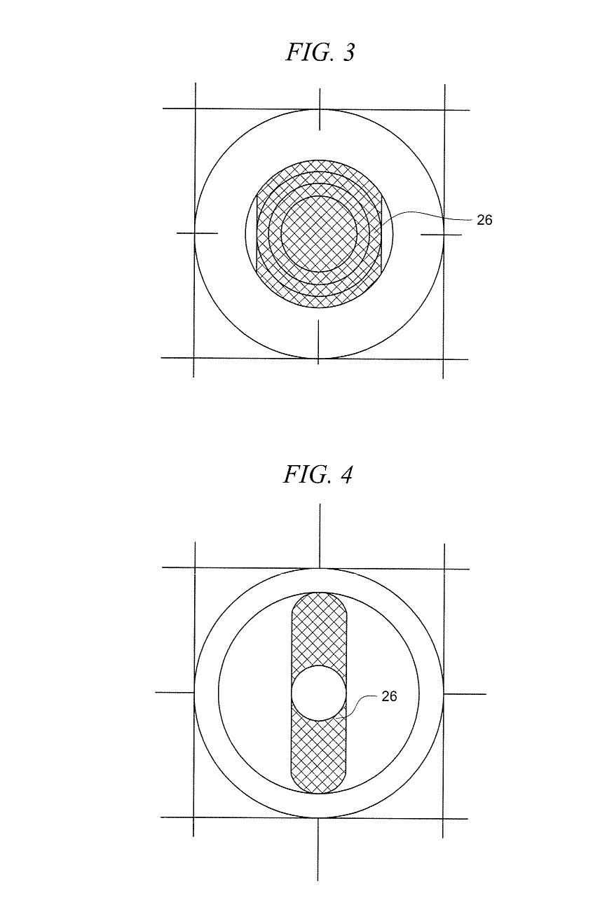

FIGS. 2A-2F depict a series of lens cross sections taken through opening 26 to show different shapes for opening 26. For example, FIG. 2A discloses an opening with the outer walls having a concave structure while FIG. 2B shows a convex structure. FIG. 3C shows the edges of the opening sloped or tapered from the outer surface to the inner surface of the lens where FIG. 2D shows the reverse. FIGS. 2E and 2F show the edge in a stepped configuration where the outer diameter of the opening is larger on the side of the lens than the opposite side of the lens. When a lifting force is being created by the fluid, the cross sectional configuration of opening 26 may be varied to change the influence of the fluid on lens 10 and to vary the flow pattern of the fluid through opening 26. FIGS. 2D and 2F may be used to create a small pocket 32 of fluid immediately before the fluid passes through opening 26. The opening size of small pocket 32 may be greater than 0.4 mm is some configurations. The inverted openings of FIGS. 2C and 2E may be used to diffuse the fluid flow as it passes through lens 10. In these examples, small pocket 32 may have a depth equal to the depth of opening 26 and opening 26 may have a diameter from 0.1 to 0.3 mm while pocket 32 has a diameter of 0.2 to 0.4 mm.

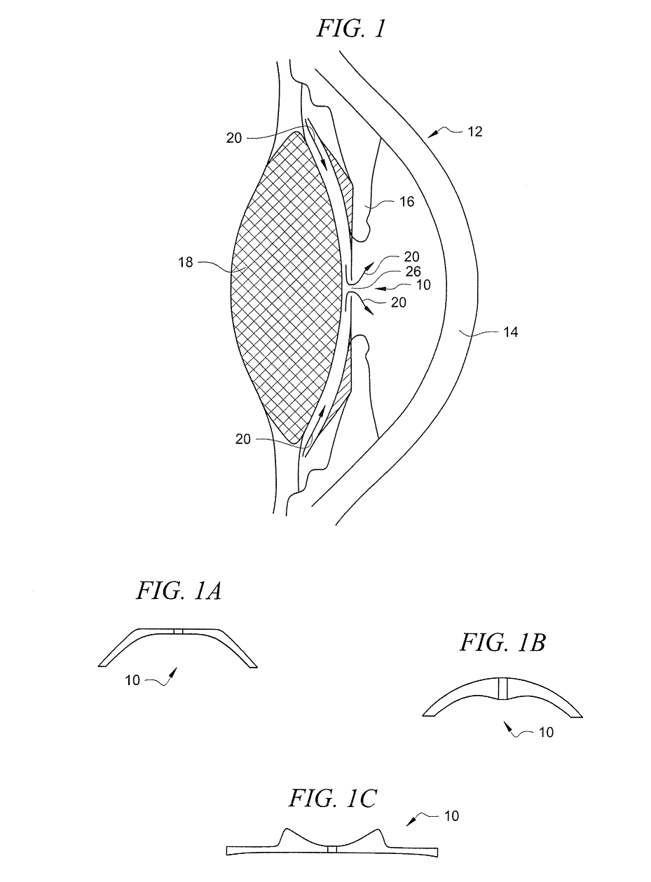

FIGS. 3 and 4 depict a series of non-circular, elongated lens openings disposed at the optical portion of lens 10. Each of these drawings locates the lengthwise dimension of lens 10 in a vertical orientation on the sheet of paper as indicated by the SI dimension. These openings may be used to even fluid flow under lens 10 because the longer dimensions of the openings are aligned with the positioning arms of lens 10. The elongated openings may thus promote fluid flow (line 20 in FIG. 1) under the positioning arms by locating the ends of opening 26 closed to the arms. As shown in the drawings, the elongated direction may be 0.1 to 0.8 mm larger than the narrow dimension. For example, the lens in FIG. 3 may have an opening with a width of 0.5 mm and a length of 0.6 mm. Similarly, the opening in FIG. 4 may have a width of 0.2 mm and a length of 0.0 mm.

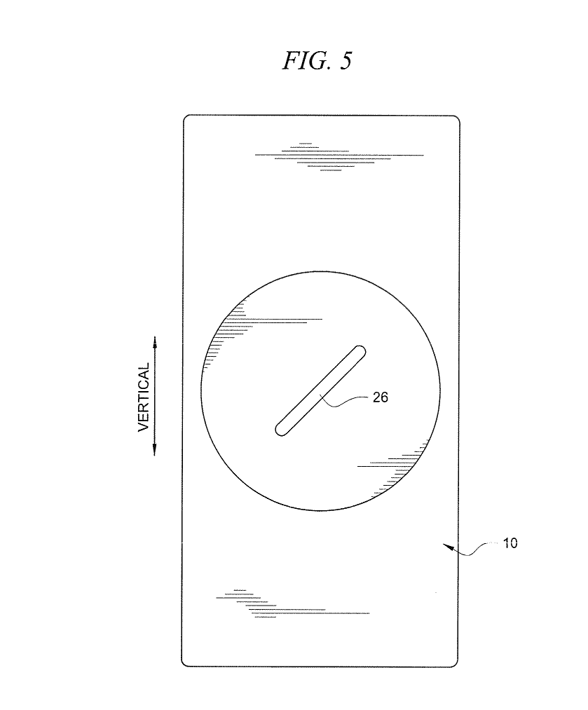

FIG. 5 shows a lens having an elongated opening 26 that may be used as a lens position indicator. Elongated opening 26 is tilted to the right such that the upper end of opening 26 is offset to the right when the lens is properly positioned in the eye. The surgeon may thus use the orientation of opening 26 to check the orientation of lens 10 during implantation. If the upper portion of opening is offset to the left with vertically-disposed positioning arms, then the surgeon knows that the lens is upside down with its posterior surface positioned adjacent the iris.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A phakic intraocular lens for implantation between the iris and the crystalline lens of the eye; the phakic lens comprising:
a lens body defining an optical portion of the lens; the lens body defining an opening at the optical portion of the lens; the opening having a maximum width dimension of less then 0.4 mm.

2. A phakic intraocular lens for implantation between the iris and the crystalline lens of the eye; the phakic lens comprising:
a lens body defining an optical portion of the lens; the lens body defining an opening at the optical portion of the lens; the opening being elongated.

3. The lens of claim 2, wherein the lens has a pair of positioning arms;
the elongated dimension of the opening being aligned with the positioning arms of the lens.

4. The lens of claim 2, wherein the ends of the openings are rounded and the sides of the opening are straight.

5. The lens of claim 4, wherein the ends of the openings are semi-circular.

6. The lens of claim 2, wherein the lens has a vertical centerline and the elongated opening is tilted with respect to the vertical centerline.

7. The lens of claim 6, wherein the top of the elongated opening is disposed to the right of the vertical centerline.

8. A phakic intraocular lens for implantation between the iris and the crystalline lens of the eye; the phakic lens comprising:
a lens body defining an optical portion of the lens; the lens body defining an opening at the optical portion of the lens;
the opening configured to create a lift force with the fluid of the eye to position to lens away from the crystalline lens of the eye.

9. The lens of claim 8, wherein the opening is tapered.

10. The lens of claim 8, wherein the opening is stepped.

11. The lens of claim 8, wherein the sidewalls that define the opening are curved in cross section.

12. A phakic lens configuration comprising: a lens body disposed in a floating relationship with respect to the iris and the crystalline lens of the eye;
the lens having an opening configured to cooperate with the fluid flow of the eye to create a force that urges the central portion of the phakic lens away from the anterior surface of the crystalline lens.

13. The lens of claim 12, wherein the opening has a maximum width dimension of 0.6 mm or smaller.
